(19) United States
(12) Patent Application Publication
(10) Pub. No.: US 2011/0189114 A1
(43) Pub. Date: Aug. 4, 2011

(54) MINOXIDIL COMPOSITION AND NON-AEROSOL, NON-SPRAY FOAM METHOD OF DELIVERY THEREFOR

(76) Inventor: Nyangenya Maniga, Gilbert, AZ (US)

(21) Appl. No.: 13/016,583

(22) Filed: Jan. 28, 2011

Related U.S. Application Data
(60) Provisional application No. 61/299,572, filed on Jan. 29, 2010.

(51) Int. Cl. A61K 31/506; A61P 17/14; A61P 17/16

(52) U.S. Cl. 424/59; 514/275

(57) ABSTRACT

A foamable composition is described that contains one or more of: minoxidil, or a pharmaceutically acceptable salt thereof, an aqueous-alcoholic solvent medium which enables said minoxidil or said pharmaceutically acceptable salt thereof to be solubilized, a surfactant, such as oleth-20, a foam stabilizer, such as lauryl glucoside, and a sunscreen. The foamable minoxidil composition is optionally delivered in a measured dosage as a foam using a non-pressurized container, where the foam is produced within the container without use of a propellant.

### CROSS-REFERENCES TO RELATED APPLICATION

This application claims the benefit of U.S. provisional patent application no. 61/299,572 filed Jan. 29, 2010, all of which is incorporated herein in its entirety by this reference thereto.

### BACKGROUND OF THE INVENTION

1. Field of the Invention

The present invention relates to a composition for administration to a human scalp for the treatment and prevention of androgenic alopecia, male and female pattern baldness.

2. Description of Related Art

Minoxidil (chemical formula 2,4-diamino-6-piperidinylpyrimidine-3-oxide) is the active ingredient of Rogaine® (New Brunswick, N.J.), which is approved by the U.S. Food and Drug Administration as a safe and effective drug. It is marketed by Pfizer for the treatment and prevention of androgenic alopecia.

Numerous investigators have demonstrated that minoxidil can stimulate visible hair growth in a majority of balding subjects. The structure and use of this compound is described in U.S. Pat. No. 4,139,619 and U.S. Pat. No. 4,596,812. Minoxidil has varying degrees of efficacy for moderating androgenic alopecia, depending on the degree of baldness, its duration, the age of the patient, and on the concentration of the drug in an appropriate delivery vehicle. There are many challenges in the topical application of pharmaceutically active agents. One major objective is to achieve percutaneous penetration of the active agent to the site of treatment. It is also desirable for the composition to have desirable cosmetic characteristics, for easy and smooth application, and for administration without a noticeable residue on the surface of the skin. It is further desirable that the composition not cause irritation or discomfort. It is still further desirable that application is convenient in terms of time requirements and in terms of necessary clean-up after application.

Topical solutions have not been satisfactory for use in treating the scalp as they tend not to remain in place long enough for satisfactory amounts of the drug to be absorbed. The solution readily runs off the site of application and therefore, it is difficult to apply controlled amounts using the minoxidil in solution form.

Formulations of minoxidil, such as jellies and ointments, have been proposed. These compositions are not pharmaceutically elegant and also are not suitable for use as treatments for stimulating the growth of hair from a cosmetic point of view. Lotion and gel topical dosage forms have the disadvantage of extended rub-in and leave oily residues.

Commercially available Rogaine®, disclosed in U.S. Pat. No. 6,946,120 includes an aerosol foam, which is filled into a pressurized container together with a propellant fluid. The composition is foamed prior to the application by means of a propellant fluid.

Aerosol containers are in widespread use as liquid dispensing devices. However, aerosol containers require the use of a pressure container and a propellant fluid to create foam. Propellants provide the appropriate vapor pressure within aerosol containers for the expulsion of the formulation as a spray or semisolid when the valve is opened. Typical propellants include liquefied petroleum gases; such as mixture of propane, isobutene, and n-butane; chlorofluorocarbons; methyl ethyl ether; and dimethyl ether, which are flammable, harmful and toxic volatile organic compounds. Further, it is known that the propellant fluid frequently constitutes a source of environmental pollution and sometimes the aerosol container represents a hazard when disposed of since the propellant material are explosive or flammable. Generally, chlorofluorocarbons are banned from use due to their ozone depletion effect, liquefied petroleum gases are flammable, and other propellants such as nitrous oxide may have physiological effects.

Still further, aerosol containers can not be completely emptied or refilled, increasing waste. Aerosol containers are discarded once the supply of liquid dispersant is exhausted or when the supply of propellant material has been used to the extent that there is no longer sufficient pressure within the container to discharge the liquid as intended. Further, pressure containers can only be cylindrical, drastically limiting the choice of container shape. An additional disadvantage is that pressure containers are more expensive than non-pressurized containers and have higher manufacturing costs.

Patents related to the current invention are summarized here.

### Aerosol Foam

T. Wai-Chiu So, et.al., “Pharmaceutical Composition”, U.S. Pat. No. 6,946,120 B2 (Sep. 20, 2005) describe a pharmaceutical composition for topical administration including at least five percent by weight of a piperidinopyrimidine derivative or an acceptable salt thereof, an acid, a solvent composition including water, a lower alcohol and co-solvents consisting of aromatic and polyhydric alcohols, where the co-solvent includes less than approximately ten percent by weight of propylene glycol.

### Foam Spray

E. Jacques, et.al., “Minoxidil Pharmaceutical Foam Formulation”, U.S. patent application no. 2005/0079139 A1 (Apr. 14, 2005) describe a pharmaceutical foam formulation in a dosage form including at least one active ingredient selected from the group consisting of minoxidil, minoxidil sulfate, other soluble minoxidil salts, a surfactant, and water; the formulation being adapted to form a foam when administered by spraying.

### Aerosol Liquid Spray

P. Cronk, et.al., “Continuous Spray Scalp Therapy and Dispensing Systems for Same”, U.S. patent application no. 2008/0206156 A1 (Aug. 28, 2008) describes continuous spray medications, spray medication dispensing systems, and methods for treating alopecia, in which a continuous mist of a scalp medication, such as minoxidil, finesteride, copper peptides, DHT inhibitors and/or androgen receptor blockers, disposed within a pharmacologically acceptable carrier solution, is administered in an amount sufficient to stimulate or maintain hair growth.

P. Uster, et.al., “Non-crystalline Minoxidil Composition”, U.S. Pat. No. 5,030,442 (Jul. 9, 1991) describe an aqueous, non-crystalline minoxidil composition for topical use containing minoxidil complexed with an amphipathic compound, oleic acid, and acceptable excipients, which has improved flux through human cadaver skin and is formulated in an aqueous vehicle or dispersed in fluorochlorocarbon solvent for spray delivery.

P. Uster, et.al., “Non-crystalline Minoxidil Composition, its Production and Application”, U.S. Pat. No. 4,828,837 (May 9, 1989) describe an aqueous, non-crystalline minoxidil composition for topical use containing minoxidil complexed with an amphipathic compound containing a single lipophilic chain moiety and a sulfate, sulfonate, phosphate and phosphonate polar moiety, and having a pK less than five. the composition is formulated in ointment form, in an aqueous vehicle, or dispersed in a fluorochlorocarbon solvent for spray delivery.

### Aqueous Vehicle

J. Cappello, “Topical and Transdermal Treatments Using Urea Formulation”, U.S. Pat. No. 7,803,357 B2 (Sep. 28, 2010) describes a topical application of a composition of urea and a chemotherapeutic agent such as sclerosing agents, vasodilators, botulinum toxin, and minoxidil that is effective in treating spider veins, erectile dysfunction, facial wrinkles, hair loss, and baldness.

S. Malek, “Topical Administration Carrier Composition and Therapeutic Formulations Comprising Same”, U.S. Pat. No. 7,749,489 B2 (Jul. 6, 2010) describes a topically administered carrier composition including water, glycerin, and polysorbate, which retards the evaporative losses of the solvent component and systemic migration losses of the active ingredient to provide sustained topical action for use in formulations containing active ingredients, such as minoxidil.

K. Hallam, et.al., “Methods and Compositions for the Promotion of Hair Growth”, U.S. Pat. No. 6,465,514 B1 (Oct. 15, 2002) describe compositions, medicaments, and methods for the promotion of hair growth, topically applied to the scalp by use of an eyedropper or other suitable means, including either local anesthetics of the secondary and tertiary amino type and/or niacin. The compositions and medicaments include minoxidil and either procaine hydrochloride or niacin or procaine hydrochloride and niacin in a non-sulfur-containing carrier.

C. Chidsey, III, et.al., “Methods and Solutions for Treating Male Pattern Alopecia”, U.S. Pat. No. 4,596,812 (Jun. 24, 1986) describe a method for treating male pattern baldness, which includes regular topical application to the human scalp of a composition containing 6-amino-1,2-dihydro-1-hydroxy-2-imino-4-piperidinopyrimidine as one of its active ingredients.

### Gel/Ointment/Cream

L. Pena, et.al., “Compositions of Minoxidil”, U.S. Pat. No. 7,442,369 B1 (Oct. 28, 2008) describe novel compositions comprising minoxidil, a thickening agent, and a pharmaceutically acceptable solvent; a process for making a gel composition including minoxidil; and methods for using the compositions for treating and preventing hair loss in a patient.

C. Samour, “Method for Treating Hair Loss”, U.S. Pat. No. 5,620,980 (Apr. 15, 1997) describes the combination of minoxidil and 2-n-nonyl-1,3-dioxolane for promoting hair growth when applied once daily.

L. Pena, “Minoxidil Gel”, U.S. Pat. No. 5,225,189 (Jul. 6, 1993) describes a pharmaceutical gel containing minoxidil for topical application.

J. Grollier, “Composition in the Form of a Gel for Inducing and Stimulating Hair Growth and for Decreasing Their Loss, Based on Piperidinopyrimidine Derivatives”, U.S. Pat. No. 4,820,512 (Apr. 11, 1989) describes a gel composition for inducing and stimulating hair growth and for reducing hair loss based on piperidinopyrimidine derivatives.

Oral in Conjunction with Topical Application

C. Catalfo, et.al., “Compositions Containing Minoxidil and Saw Palmetto for Treating Baldness”, U.S. Pat. No. 6,596,266 B2 (Jul. 22, 2003) describe compositions containing minoxidil as an active ingredient, other active agents and/or enhancer agents, such as saw palmetto extract and nettle root extract, and methods of using the compositions to treat male patterned baldness and to stimulate hair growth on the scalp.

### Encapsulation

N. Weiner, et.al., “Stimulation of Hair Follicles”, U.S. Pat. No. 5,834,014 (Nov. 10, 1998) describe a novel method and delivery system for the topical delivery of a therapeutic weak acid or a base material, such as minoxidil, that utilizes a therapeutic material, which is modified to make it more hydrophilic, encapsulated in a lipid vesicle, preferably a non-phospholipid vesicle.

### Problem

There remains in the art a need for an effective formulation and/or delivery method for delivery of minoxidil and/or aminoxidil salt.

### SUMMARY OF THE INVENTION

The invention comprises aminoxidil composition or a salt thereof and foamable non-aerosol, non-spray delivery thereof.

### BRIEF DESCRIPTION OF THE DRAWINGS

A more complete understanding of the present invention is derived by referring to the detailed description and claims when considered in connection with the Figures, wherein like reference numbers refer to similar items throughout the Figures.

FIG. 1 illustrates key features of a foamable non-aerosol, non-spray product composition and delivery system;

FIG. 2 illustrates formulation of the foamable non-aerosol, non-spray product;

FIG. 3 illustrates schematically an aerosol foam;

FIG. 4 illustrates schematically a non-aerosol, non-spray foam; and

FIG. 5 illustrates a non-pressurized delivery container delivering a foam.

### DETAILED DESCRIPTION OF THE INVENTION

The invention comprises a non-aerosol, non-spray foam product comprising the active ingredient minoxidil or a pharmaceutically acceptable salt thereof, for the treatment and prevention of androgenic alopecia, male and female pattern baldness.

In one embodiment, a foamable composition includes: approximately one-half percent or greater minoxidil, or a pharmaceutically acceptable salt thereof, by weight, based on total weight of the foamable composition; a solvent system, the solvent system comprising an aqueous-alcoholic solvent medium which enables the minoxidil or the pharmaceutically acceptable salt thereof to be solubilized; a surfactant, the surfactant comprising oleth-20; and a foam stabilizer, the foam stabilizer comprising lauryl glucoside.

In another embodiment, a foamable non-aerosol, non-spray product containing minoxidil or a salt thereof as the pharmaceutically active compound, which is useful for the treatment and prevention of androgenic alopecia is delivered from a non-pressurized container. A foam generated within the container is dispensed in a measured dosage as a liquid foam administered topically to the human scalp.

Hereinafter, for clarity the active ingredient is referred to as minoxidil. However, all formulations and all delivery methods described herein additionally apply to delivery of a pharmaceutically acceptable salt of minoxidil as the active ingredient.

### Advantages

Delivery of a measured dosage of a foam product directly to the human scalp from the container has a number of advantages, including:
easy and smooth delivery of the product directly to the scalp;
delivery of a measured dosage of the formulation comprising minoxidil or a salt thereof;
economic delivery of the formulation; and
delivery from a non cylindrical container.

The foam is non-runny, easy to apply, breaks easily with shear, and uses a low residue delivery vehicle. When the foam is applied, body heat causes the foam structure to break down and deposit the active ingredient in the form of a vehicle resembling a solution. The foam composition is light, gentle, and easy to control on application to the desired area of the scalp. The foam allows controlled and precise delivery of a dosage of the active ingredient to the scalp without an intermediate transfer container, a hand, and/or the running of the active agent off of the scalp in a liquid deliver vehicle.

### Foamable Liquid Composition

The foamable liquid composition optionally includes:
minoxidil or a pharmaceutically acceptable salt thereof as the active ingredient;
a solvent system;
a surfactant; and
a stabilizer.

Each of the minoxidil, the solvent system, the surfactant, and the stabilizer are further described, infra.

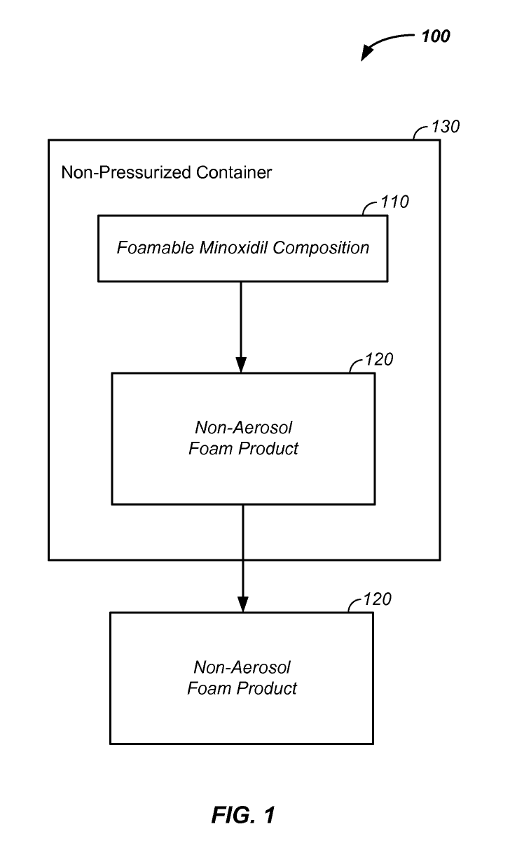

Referring now to FIG. 1, a foam delivery method 100 of a non-aerosol, non-spray foam product 120 includes: a foamable minoxidil composition 110 held in a non-pressurized container 130 and a method of creating the non-aerosol, non-spray foam product 120 within the container 130 and dispensing the non-aerosol, non-spray foam product 120 from the container 130.

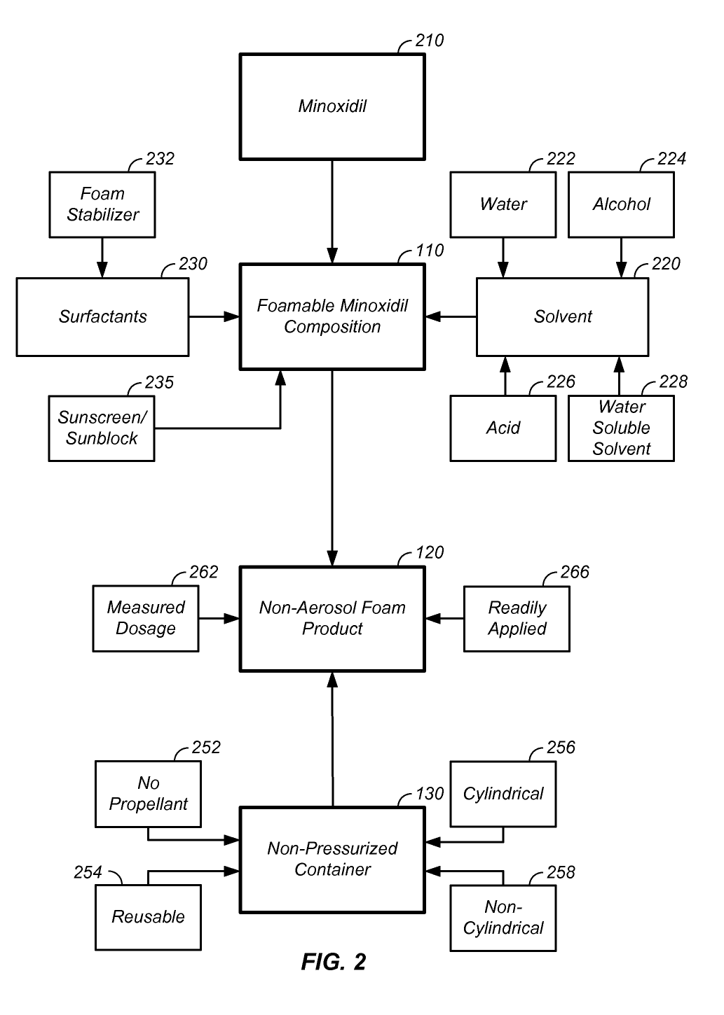

Referring now to FIG. 2, details of use of the non-pressurized container 130 to dispense the foamable minoxidil composition 110 as the non-aerosol foam product 120 for the treatment and prevention of hair loss in humans are provided. The foamable minoxidil composition is preferably dispensed using non-aerosol, non-spray means.

### Foamable Liquid Formulation

Still referring to FIG. 2, details of the foamable minoxidil composition 110 are provided. The foamable minoxidil composition 110 optionally includes one or more of:
a surfactant 230, where the surfactant optionally includes:
   a foam stabilizer 232;
a solvent 220, such as
   water 222;
   an alcohol 224;
   an acid 226; and
   a water soluble solvent 228; and
a sunscreen and/or a sun block 235.

### Minoxidil

Minoxidil 210 or a pharmaceutically acceptable salt thereof is preferably used in portions of between one-half and ten percent by weight and preferably in portions of between two and five percent by weight relative to the total weight of the composition.

### Solvent

The foamable minoxidil composition is preferably in a solvent 220. Preferably the solvent 220 is an aqueous-alcoholic medium, which enables solubilization of the minoxidil 210. In a first example, the solvent 220 includes water 222 from twenty to eighty percent by weight. Preferably the water 222 makes up thirty to sixty percent of the foamable minoxidil composition 110 by weight.

In an first example, the solvent 220 includes an acid 226 at a concentration of one-half to five percent by weight of the foamable minoxidil composition 110. The acid 226 is optionally any inorganic acid, any organic acid with chain length of eight carbons or less, or a molecule containing eight carbons or less. A preferred composition of the solvent 220 includes:
lactic acid in proportions between about one and four percent by weight;
a lower alcohol 224 with one to four carbon atoms, such as methanol, ethanol, propanol and isopropyl alcohol, preferably ethanol in proportions of between about one-tenth and fifty percent by weight and more preferably between about five and thirty percent by weight; and
one or more water soluble solvents 228, such as butylene glycol, glycerin, polyglycerin, ethylene glycol, and propylene glycol, preferably propylene glycol in proportions of between about zero and twenty percent by weight, and more preferably between about five and fifteen percent by weight,
   where all of the weights are relative to the total weight of the foamable minoxidil composition 110.

### Surfactant

Foam is produced by the introduction of air or other gas into a liquid phase, during which time the bubbles become encapsulated in a film of the liquid. The thin liquid film separating two or more gas bubbles is referred to as a lamellar film.

For a liquid to form foam, it must be able to form a membrane around the gas bubbles possessing a form of elasticity that opposes the thinning of the lamellae as a result of loss or drainage of the liquid. Foaming does not occur in pure liquids because no such mechanism for the retardation of lamellae drainage or interfacial stabilization exists. However, when surface-active substances, such as a surfactant, is present their adsorption at the gas-liquid interface serves to retard the loss of liquid from the lamellae and in some instances, to produce a more mechanically stable system.

The formation and persistence of foams under various conditions is explained by the complimentary effects known as the Gibbs-Marangoni effects. The Gibbs effect states that the surface tension of a solution will decrease as the concentration of the surfactant in solution increases until the critical micelle concentration (cmc) is achieved. The Marangoni effect states that there is a finite, diffusion rate based, time during which the surface-active molecules in the bulk solution diffuse to the interface to lower the surface tension of a newly formed surface.

Therefore, the ability of a surfactant to perform as a foaming agent is dependent primarily on its effectiveness at reducing the surface tension of the solution, the rate of diffusion of the surfactant molecules in the bulk solution to the interface to reduce the surface tension, the rate of adsorption of the surfactant molecules at the interface, its properties with regard to surface pressures or disjoining pressures due to overlapping of the surface layers in thin films, and the elastic properties it imparts to interfaces.

Hence, the effectiveness of a surfactant is determined by how the surfactant interacts with the other constituents of the system in question. Hence, a custom-built surfactant formulation is herein presented to achieve a combination of surfactant actions to suit the individual needs of the system.

A preferred embodiment of the liquid foam according to the invention contains at least one surfactant 230. The surfactant 230 is preferably contained in an amount of one-tenth to five percent by weight of the foamable minoxidil composition 110, and more preferably is in the range of about two-tenths to one percent by weight of the foamable minoxidil composition 110. Suitable surfactants have emulsifying, solvating, and foam-forming or foam-stabilizing properties; are preferably nonionic; and have a hydrophilic-lipophilic balance (HLB) value of greater than about fifteen. In particular, the surfactant oleth-20 is preferred in proportions between about one tenth and five percent by weight of the foamable minoxidil composition 110 and more preferably between about two-tenths and one percent by weight relative to the total weight of the foamable minoxidil composition 110.

Other surfactants optionally used with the present formulation include, but are not limited to: any combination of anionic, cationic, non-ionic, zwitterionic, or amphoteric surfactants and non-ionic block copolymers with an HLB value of greater than fifteen.

### Stabilizer

Optionally, the non-aerosol foam product 120 is maintained with a foam stabilizer 232. In the application of treatment of the human scalp for androgenic alopecia the maintenance of foam is important to allow a known and suitable period of contact of the minoxidil 210 to the scalp.

Lamellar films between adjacent bubbles can be easily stretched as a result of gravity, agitation, drainage, and other motion leading to collapsing of the foam. In spite of their tendency to collapse, however, foams can be prepared that have a lifetime or persistence of minutes, days, or even months. Low persistence foams remain for a very short time and collapse due to overwhelming effects of surface tension and gravitational forces. More persistence foams can be produced by introduction of small amounts of amphiphilic substances or a foam stabilizer 232 to the aqueous system.

The foam stabilizer 232 alters the characteristics of the aqueous system and hence enhances the stability of the foam and extends the lifetimes as a result of one or several of the following: (1) increasing the viscosity in the liquid phase, slowing drainage of the liquid from between the bubble interfaces, as well as providing a cushion effect to absorb shocks resulting from random or induced mechanical disturbances/motion; (2) increasing the surface viscosity, which also retards liquid loss from between interfaces by a viscous drag type of mechanism; (3) enhancing surface effects such as the Gibbs and Marangoni effects, which act to “heal” areas of film thinning due to loss or drainage of liquid; and (4) electrostatic and steric repulsion between adjacent interfaces due to the adsorption of ionic and nonionic surfactants, polymers, or other compounds. The foam stabilizer 232 also has the effect of lowering the surface tension of the system, which reduces the work required for the initial formation of the foam.

A preferred embodiment of the foamable minoxidil composition 110 contains at least one stabilizer 232. The stabilizer 232 is preferably contained in an amount of about 0.05 to 0.5 percent, and more preferably from one tenth to five tenths percent by weight. In particular, the stabilizer includes lauryl glucoside with a portion between about 0.05 and 0.5% by weight and more preferably between one-tenth and five-tenths percent by weight relative to the total weight of the composition.

Other optional foam stabilizers 232 used with the present formulation include, but are not limited to: any fatty amine oxide, a quaternary amine, or a cellulose derivatives, such as methyl cellulose and ethyl cellulose.

It is a principal object to provide a liquid foamable minoxidil composition 110 or formulation for topical administration to the scalp where:
the active ingredient comprises minoxidil 210 or a pharmaceutically acceptable salt thereof, preferably between about one-half and ten percent by weight and more preferably between about two and five percent by weight relative to the total weight of the foamable minoxidil composition 110;
the solvent 220 or aqueous medium, which enables the minoxidil to be solubilized, preferably includes:
   water 222 from about twenty to eighty percent by weight and more preferably from about thirty to sixty percent relative to the total weight of the foamable minoxidil composition 110;
   acid 226 from about one-half to five percent by weight, such as any inorganic acid or any organic acid with eight carbons or less, preferably lactic acid between about one and four percent by weight of the foamable minoxidil composition 110;
   a lower alcohol with one to four carbon atoms, such as methanol, ethanol, propanol, and isopropyl alcohol, preferably ethanol in proportions of between about one-tenth and fifty percent by weight and more preferably between about five and thirty percent by weight of the foamable minoxidil composition 110; and
   a water soluble solvent 228, such as butylene glycol, glycerin, polyglycerin, ethylene glycol, and propylene glycol, preferably propylene glycol in proportions of between about zero and twenty percent by weight and more preferably between about five and fifteen percent by weight; and
at least one surfactant 230, such as any combination of anionic, cationic, non-ionic, zwitterionic surfactant, and non-ionic block copolymers with an HLB value of greater than about fifteen, preferably oleth-20 in proportions of about one-tenth to five percent by weight and more preferably from about two-tenths to one percent by weight of the foamable minoxidil composition 110, and
   at least one foam stabilizer 232, such as any fatty amine oxides, quaternary amines, or cellulose derivatives, such as methyl cellulose and ethyl cellulose, but preferably lauryl glucoside in proportions of about 0.05 to 0.5 percent by weight and more preferably from about one-tenth to one-half percent by weight of the foamable minoxidil composition 110.

### Method of Dispensing Non-Aerosol, Non-Spray Foam

Another object relates to the dispensing of stable non-aerosol, non-spray foam that breaks easily with shear as the vehicle system for dispensing out the liquid formulation comprising the active ingredient minoxidil or a pharmaceutically acceptable salt thereof.

### Aerosol Foam

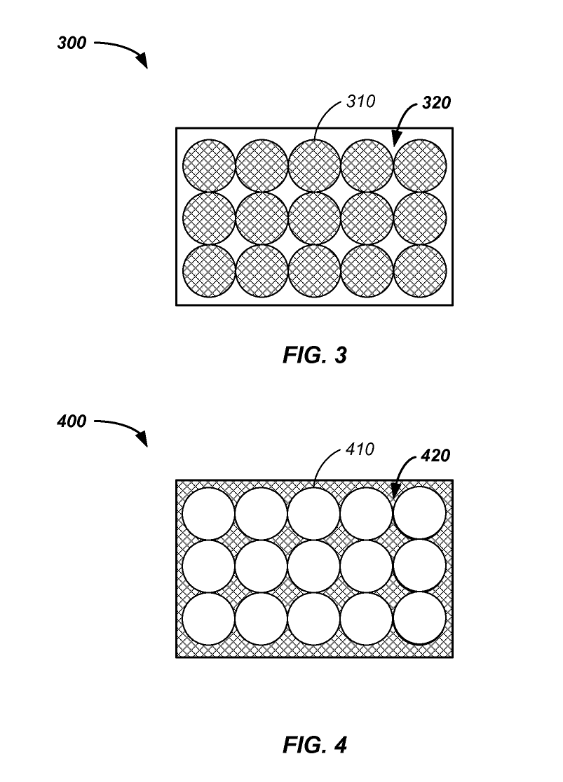

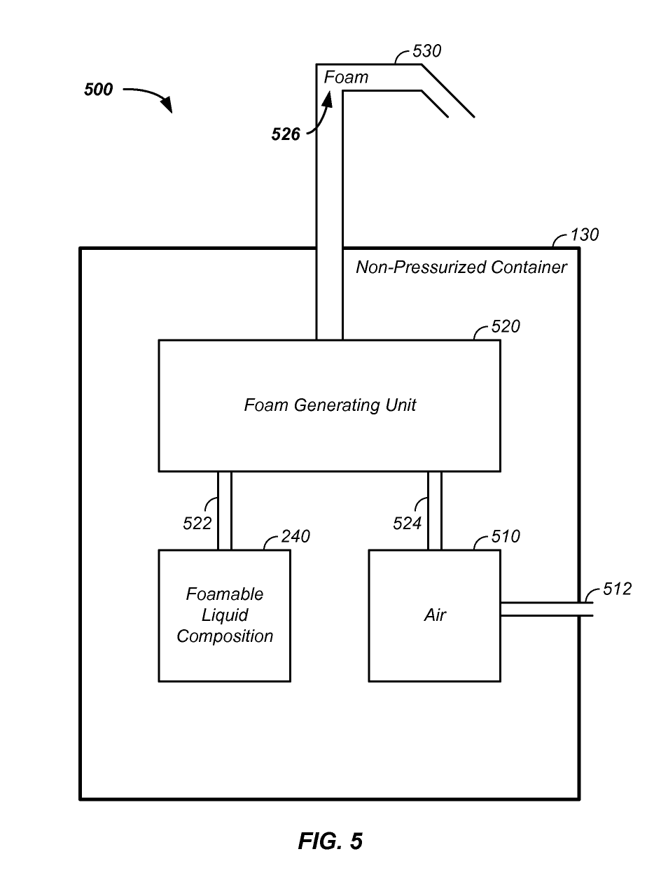

Referring now to FIG. 3, aerosol foam 300 is depicted illustratively. The term “aerosol foam” is a product, which includes a liquid foamable composition and a propellant liquid, filled into a pressurized container that is equipped with a valve system and nozzle at the top of the container, and a dip tube that runs from the valve system to the bottom of the container. When the valve is open, the pressure on the liquid propellant is instantly reduced and it starts to evaporate forming a high-pressure gas layer at the top of the container. This high-pressure gas layer pushes the liquid product, as well as some of the liquid propellant, up the dip tube and out through the nozzle. When the liquids flow through the nozzle, the liquid propellant evaporates into gas and in the process forms propellant gas bubbles 310 in the liquid product 320 creating foam.

### Non-Aerosol Non-Spray Foam

Referring now to FIG. 4, non-aerosol, non-spray foam 400 is depicted illustratively. The clause “non-aerosol, non-spray foam” is a product, which comprises a foamable liquid composition 420, which is filled into a non-pressurized container that is equipped with a mechanical pump, an air chamber, a mixing chamber, a foam forming screen mesh, and a dip tube that runs from the mechanical pump to the bottom of the container. When the mechanical pump is actuated, the foamable liquid composition 420 is forced up the dip tube into the mixing chamber where it is comingled with air from the air chamber, at a predetermined ratio to form foam. It is the actuation of the pump that pressurizes and causes the turbulent comingling of the air and the liquid foamable composition 420 to form air bubbles 410 in the foamable liquid composition 420 creating foam.

From the mixing chamber, the foam is then homogenized into fine uniform bubbles when it passes through the screen mesh before being dispensed out through the nozzle. Therefore, it is a non-propellant method of propelling the liquid product out of the container in the form of foam.

In summary, a major difference in the continuous state of the containers is that in an aerosol foam container the propellant fluid (typically compressed gas or liquefied gas) is pumped into the container under high pressure after the container is sealed and is maintained continuously in pressurized state, whereas in stark contrast in the container of the non-aerosol, non-spray foam, the air inside the container is constantly under atmospheric pressure.

### Non-Aerosol, Non-Spray Foam Pump and Container

In still yet another embodiment, a final product of a liquid formulation comprising the active ingredient minoxidil or a pharmaceutically acceptable salt thereof, that is packaged in a non-pressurized container 130, is dispensed using a non-aerosol, non-spray foam pump. The non-aerosol, non-spray foam pump provides for safe and simple dispensing of a measured dosage 262 of the foam that contains the liquid formulation comprising minoxidil or a salt thereof, that is readily applied 266 (breaks easily with shear) to the scalp. The non-aerosol, non-spray foam pump is calibrated to deliver an adequate volume of the foam.

An additional object herein disclosed is to provide the liquid formulation in a reusable 254 and non-pressurized container 130 that can be manufactured in a cylindrical 256 or non-cylindrical 258 shapes and that contains no propellant 252.

Therefore, the present embodiment does not employ the use of pressurized containers containing typical propellants, such as liquefied petroleum gases (mixture of propane, isobutene, and n-butane), chlorofluorocarbons (CFCs), and dimethyl ether, which are flammable, harmful, and toxic volatile organic compounds (VOCs). The present embodiment is safely transported, stored, and dispensed in reusable containers. Reusing products and the parts of products is extremely important for the safety and future of our planet.

Yet another aspect of the present embodiment relates to the orientation of the container when the product is being expelled; the foam can be easily and conveniently expelled in both horizontal (upright) and vertical positions.

Still another objective relates to the dispensing of a measured dosage of the liquid formulation; the container is calibrated to deliver an adequate volume of the foam that contains the liquid formulation comprising the active ingredient minoxidil or a pharmaceutically acceptable salt thereof.

Other objects are to provide a method for treating and/or preventing hair loss in humans, which is safe, simple, painless, cosmetic in the sense of being invisible, and easy to apply, where the methods comprise topically administering to the human scalp compositions.

### Sunscreen

In still yet another embodiment, a protection agent is provided in the foamable minoxidil composition 110. The protection agent is delivered to a balding scalp in the foam and yields protection from the damaging effects of UV-A and/or UV-B irradiation, particularly solar radiation. Sunlight or ultraviolet radiation in the UV-B range has a wavelength of 290 nm to 320 nm and is known to be the primary cause of erythema, a reddening of the skin also known as sunburn. While, ultraviolet rays at a wavelength of 320 nm to 400 nm, known as UV-A radiation, produces tanning of the human epidermis. UV-A rays can also cause a loss in the elasticity of the skin and the appearance of wrinkles leading to a premature aging of the skin.

In this embodiment the minoxidil liquid formulation 110 includes a sunscreen and/or a sun block agents, topically administered to the scalp in the cosmetically acceptable delivery vehicle of the non-aerosol, non-spray foam. The sunscreen and/or sun block agents 235 in the minoxidil liquid formulation 110 include at least one of: an organic chemical compound that absorbs ultraviolet light, an inorganic particulate that reflects, scatters, and absorbs ultraviolet light, and an organic particulate that absorbs, reflects, and scatters ultraviolet light. Preferably, the sunscreen and/or sun block agents 235 yields a sun protection factor (SPF) in conjunction with the delivered foam of at least 1, 2, 3, 4, 5, 10, 15, 20, 30, or 40 to the scalp in the dosage of foam delivered for a unit dosage of minoxidil 210.

In yet another embodiment, a foam dispensing device for dispensing the foamable minoxidil composition 110 as a foam includes one or more of: a non-pressurized container for holding the foamable minoxidil composition 110, a foam generating unit, an air chamber, a liquid chamber, and an outflow channel through which the foamed liquid product exits the foam dispenser through the nozzle. The liquid chamber optionally includes a liquid inlet valve and a liquid discharge valve. The air chamber also optionally includes an air inlet valve and an air discharge valve. The non-pressurized container need not be cylindrical. The foam dispensing device, optionally further includes a dip tube, which draws the foamable liquid composition from the bottom of the non-pressurized container 130 though the liquid chamber and into the foam generating unit.

Operation of the dispensing foam occurs in the following manner. When downward pressure is applied against the nozzle, the air in the air chamber is compressed and forced through the air discharge valve into the foam generating unit. Concurrently, the foamable minoxidil composition 110 in the liquid chamber is forced through the liquid discharge valve into the foam generating unit, where the turbulent comingling of the air and foamable minoxidil composition 110 creates foam. The resulting foam is expelled through the foam generating discharge valve into the outflow channel and out through the nozzle opening.

Once the downward pressure on the nozzle is released, the resulting suction in the liquid chamber forces open the liquid inlet valve, allowing the foamable minoxidil composition 110 to be suctioned through the dip tube into the liquid chamber. The liquid discharge valve remains closed during this return stroke. Concurrent with the release of the downward pressure on the nozzle, air is drawn into the air chamber via the air inlet valve, bringing the pressure above the foamable minoxidil composition 110 in the non-pressurized container 130 back to atmospheric pressure.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. A liquid for the treatment and prevention of hair loss in humans, said liquid comprising:
a foamable composition, said foamable composition comprising:
approximately one-half percent or greater minoxidil, or a pharmaceutically acceptable salt thereof, by weight, based on total weight of said foamable composition;
a solvent system, said solvent system comprising an aqueous-alcoholic solvent medium which enables said minoxidil or said pharmaceutically acceptable salt thereof to be solubilized;
a surfactant, said surfactant comprising oleth-20; and
a foam stabilizer, said foam stabilizer comprising lauryl glucoside.

2. The liquid of claim 1, said solvent system comprising:
water;
an acid, said acid comprising at least one of:
an inorganic acid; and
an organic acid comprising eight or fewer carbon atoms per molecule;
a lower alcohol comprising one to four carbon atoms per molecule; and
a water soluble solvent.

3. The liquid of claim 2, said lower alcohol comprising at least one of:
methanol;
ethanol;
propanol; and
isopropyl alcohol.

4. The liquid of claim 3, said water soluble solvent comprising at least one of:
butylene glycol;
glycerin;
polyglycerin;
ethylene glycol; and
propylene glycol.

5. The liquid of claim 4, said minoxidil or pharmaceutically acceptable salt thereof comprising an amount of approximately one-half to ten percent by weight of said foamable composition.

6. The liquid of claim 5, said minoxidil or pharmaceutically acceptable salt thereof comprising approximately two to five percent by weight of said foamable composition.

7. The liquid of claim 2, said acid comprising:
lactic acid.

8. The liquid of claim 7, said lower alcohol comprising:
ethanol.

9. The liquid of claim 8, said water soluble solvent comprising:
propylene glycol.

10. The liquid of claim 1, further comprising at least one of:
a protective agent, comprising:
a sunscreen; and
a sun block agent,
wherein said protective agent in conjunction with the foamable composition yields a sun protection factor of at least five,
wherein at least one of said sunscreen and said sun block agent comprise:
an organic chemical compound that absorbs ultraviolet light;
an inorganic particulate that reflects, scatters, and absorbs ultraviolet light; and
an organic particulate that absorbs, reflects, and scatters ultraviolet light.

11. The liquid of claim 1, said foamable composition comprising:
at least one of said minoxidil and said pharmaceutically acceptable salt of minoxidil comprising one-half to ten percent by weight of said liquid;
water at about twenty to eighty percent by weight of said liquid;
lactic acid at about one-half to five percent by weight of said liquid;
ethanol at about one-tenth to fifty percent by weight of said liquid;
propylene glycol at about zero to twenty percent by weight of said liquid;
said oleth-20 at about one-tenth to five percent by weight of said liquid; and
said lauryl glucoside at about 0.05 to 0.5 percent by weight of said liquid.

12. A liquid for the treatment and prevention of hair loss in humans, said liquid comprising:
a foamable composition, said foamable composition comprising:
approximately one-half percent or greater minoxidil, or a pharmaceutically acceptable salt thereof, by weight, based on total weight of said foamable composition;
a solvent system, said solvent system comprising an aqueous-alcoholic solvent medium;
a foam stabilizer, said foam stabilizer comprising at least one of:
a fatty amine oxide;
a quaternary amine;
a derivative of cellulose;
a methyl cellulose derivative; and
an ethyl cellulose derivative; and
a surfactant comprising at least one of:
an anionic surfactant;
a cationic surfactant;
a non-ionic surfactant;
a zwitterionic (amphoteric) surfactant; and
a non-ionic block copolymer,
wherein the foamable composition comprises a hydrophilic-lipophilic balance value of greater than fifteen.

13. The liquid of claim 12, said surfactant comprising:
a polysorbate.

14. The liquid of claim 12, said surfactant comprising:
oleth-20.

15. The liquid of claim 12, said foam stabilizer comprising:
lauryl glucoside.

16. The liquid of claim 12, further comprising at least one of:
a protective agent, comprising:
a sunscreen; and
a sun block agent,
wherein said protective agent in conjunction with the foamable composition yields a sun protection factor of at least two,
wherein at least one of said sunscreen and said sun block agent comprise:
an organic chemical compound that absorbs ultraviolet light;
an inorganic particulate that reflects, scatters, and absorbs ultraviolet light; and
an organic particulate that absorbs, reflects, and scatters ultraviolet light.

17. The liquid of claim 12, comprising:
at least one of said minoxidil and said pharmaceutically acceptable salt of minoxidil comprising two to five percent by weight of said liquid;
water at about thirty to sixty percent by weight of said liquid;
lactic acid at about one to four percent by weight of said liquid;
ethanol at about five to thirty percent by weight of said liquid;
propylene glycol at about five to fifteen percent by weight of said liquid;
oleth-20 at about two-tenths to one percent by weight of said liquid; and
lauryl glucoside at about one-tenth to one-half percent by weight of said liquid.

18. A method of delivering a liquid as a foam for the treatment and prevention of hair loss in humans, said method comprising the steps of:
providing a non-pressurized container, said container comprising:
a foamable composition, said foamable composition comprising:
approximately one-half percent or greater minoxidil, or a pharmaceutically acceptable salt thereof, by weight, based on total weight of said foamable composition;
a solvent system, said solvent system comprising an aqueous-alcoholic solvent medium which enables said minoxidil or said pharmaceutically acceptable salt thereof to be solubilized;
a foam stabilizer; and
a surfactant;
producing within said container a foam state of said foamable composition without use of a propellant; and
dispensing a measured dosage of said foam state of said foamable composition from said non-pressurized container.

19. The method of claim 18, wherein said step of producing further comprises a step of:
mixing said foamable composition and non-pressurized air, the air contained within said container,
wherein said foamable composition comprises oleth-20.

20. The method of claim 18, wherein said step of producing further comprises a step of:
mixing said foamable composition and air, the air at a non-pressurized state prior to said step of mixing, the air contained within said container,
wherein said foamable composition comprises lauryl glucoside.
